(19) United States
(12) Patent Application Publication
(10) Pub. No.: US 2010/0010613 A1
(43) Pub. Date: Jan. 14, 2010

(54) ARTERIOVENOUS FISTULA

(75) Inventor: Jurgen Dorn, Neulussheim (DE)

Correspondence Address: Rutan & Tucker, LLP., 611 ANTON BLVD, SUITE 1400, COSTA MESA, CA, 92626, US

(73) Assignee: C.R. Bard, Inc., Murray Hill, NJ (US)

(21) Appl. No.: 12/438,357

(22) PCT Filed: Aug. 30, 2007
(86) PCT No.: PCT/EP07/59044

§ 371 Date: Feb. 20, 2009

(30) Foreign Application Priority Data
Aug. 30, 2006 (GB) 0617074.0

(51) Int. Cl. A61F 2/06

(52) U.S. Cl. 623/1.11; 623/1.15; 623/1.34

(57) ABSTRACT

A covered radially-expansible stent (50), capable of being advanced along a bodily lumen, and capable of performing as an arteriovenous shunt, and with a covering that stops short of one end of the stent at a line that slants to the longitudinal axis of the stent at an angle intermediate between 0° and 90°.

### TECHNICAL FIELD

This invention relates to a shunt for providing an arteriovenous fistula, a system for placing the shunt and a method for placing the shunt.

Individuals suffering from renal failure undergo haemodialysis, in which a dialysis machine performs the function of the failed kidneys. Haemodialysis has to be repeated periodically and frequently and this requires repeated puncturing of the arteriovenous system using relatively large diameter dialysis needles. Frequent puncturing of native vessels with large bore needles can cause trauma and eventually a loss of patency of those vessels.

### BACKGROUND ART

It has previously been proposed to provider externally of the body, an arteriovenous shunt that can be accessed with a dialysis needle, thereby to relieve the individual of the adverse effects of repeated puncturing of native vessels. However, there remains the problem of reliable sealing of the synthetic shunt wall, and the risks of infection arising from the percutaneous nature of the synthetic shunt. It is an object of the present invention to mitigate these disadvantages.

JP 2003-245343 aims to create a subcutaneous shunt structure for dialysis, using a self-expanding stent, bare at its ends and covered in a mid-length portion that shunts between a vein and an artery in the forearm of a patient. The shunt is to be placed using a hollow hypodermic needle which is advanced through the vein and into the artery. A guidewire is to be advanced through a bore of the needle and beyond. The needle is to be withdrawn, leaving the guidewire in place. A balloon device is then to be advanced over the wire and, when its leading end is in the artery, it is to be inflated to make room for the shunt, then deflated and retracted. Then, a covered stent, catheter-based, delivery system is to be advanced over the guidewire till the stent shunt is in position, then an outer sheath around the stent is withdrawn, to release the self-expanding stent, thereby installing the shunt. The catheter and the guidewire are then to be removed.

WO 02/02163 discloses creating a fistula between a vein and an artery using a catheter in the vein and another in the artery. One of the catheters delivers a stent to the site of the fistula and places it in the vein. The procedure is for arterializing a vein, in treatment of peripheral vascular disease, as a means of avoiding amputation of the diseased limb, for example, the foot.

WO 2007/087368 has a similar disclosure, with magnets to bring together the venous and arterial catheters together.

WO 2005/000165 discloses a stent covered with inner and outer layers of PTFE with a marker sandwiched between the two layers.

WO 2005/044076 discloses an expandable prosthesis equipped with a visual maker to be viewed by an endoscope.

U.S. Pat. No. 6,264,684 discloses self-expanding stent grafts. The graft material can be expanded PTFE and the stent material can be a shape memory alloy.

WO 98/34676 discloses a subcutaneous arteriovenous fistula with a needle-receiving access site to be punctured by a needle for haemodialysis, within the context of the known Squitieri haemodialysis and vascular access system.

### SUMMARY OF THE INVENTION

In a first aspect, this invention provides a covered radially expansible stent, capable of being advanced along a bodily lumen, and capable of performing as an arteriovenous shunt. The shunt is one that is “stickable”, that is to say, it can endure repeated puncturing with a dialysis needle. The shunt is located subcutaneously, so that the only element that is percutaneous during dialysis is the dialysis needle. One way to enhance sealing of the wall of the shunt, when dialysis is completed and the needle is withdrawn, is to provide a special gel between the puncture site and the skin overlaying it. The special gel may be the one marketed under the trademark EGRESS. The shunt resembles a stent graft, known per se, in which a metal stent matrix of struts is covered by a thin membrane of expanded polytetrafluoroethylene (ePTFE).

After placement, one end of the shunt lies inside an artery, and the other end of the shunt lies inside a vein. It will be appreciated that there is a blood pressure differential across the ends of the shunt and that the shunt end in the artery is at the high pressure end of the shunt. The known shunt has the shape of a loop that bends through about 180°, so that one of its open ends faces upstream in the artery and the other of its open ends faces downstream in the vein. However, with the present invention, it is contemplated that the shunt, after placement, will take a more or less straight line in its intermediate section bridging between the vein and the artery, its one open end facing downstream in the artery and its other open end also facing downstream, but in the vein.

Preferably, the cover on the stent does not extend all the way to the ends of the stent matrix. The uncovered end section of the stent matrix, at each end of the stent, serves as an anchor for the shunt, within the lumen of the respective artery and vein. Since the shunt should extend across from the artery to the vein, along a line that takes an acute angle with respect to the upstream length direction of the artery, the covering of the stent should stop short of the end of the stent inside the artery, at a line that is slanting with respect to the long axis of the stent, with the objective that the portion of the shunt that lies within the arterial lumen is an uncovered portion (whereby blood can flow through the open interstices of the stent mesh anchoring the shunt inside the lumen of the artery). At the downstream end of the shunt, where the stent is in the lumen of the vein, it is less important that the end section of the stent matrix is uncovered but, if it is, it is preferred that the end of the covering should be more or less aligned with the wall surface of the arterial lumen.

For placement of the shunt (as described below) it will be advantageous to equip the stent with at least one radiopaque marker and there may well be benefit in providing the stent with at least one visual marker, to be viewed directly by the unaided human eye, also as explained below. The visual marker will conveniently be of a strong colour, that stands out relative to adjacent areas of the stent. Since a covering of ePTFE is usually of a white colour, there is full scope for a range of coloured markers to indicate different parts of the stent.

Importantly, the system of the present invention contemplates delivering the arteriovenous shunt trans-luminally. It is characterised by a catheter for advancing a covered stent carried near the distal end of the catheter along a bodily lumen into a position in which the stent can function as an arteriovenous shunt between a first and a second bodily lumen, the catheter including means to puncture (that is to say, to broach) radially outwardly a wall of said first lumen and radially inwardly a wall of said second lumen, and then deploy the stent.

The way in which the arterial wall and the venous wall is broached can be by means of a penetrating point provided at the distal end of the catheter and, normally, the catheter includes means by which an operator at the proximal end of the catheter can move the point axially, between a distally advanced unsheathed penetrative disposition for penetrating first the wall of the artery and then the wall of the vein, and a proximally retracted sheathed non-penetrative disposition, in which the point will be located during the period when the catheter is advanced to the point where it is to broach the vascular wall.

Since the catheter will lie along the axis of the bodily lumen, yet is required to broach the wall of the lumen in which the catheter advances, the penetrative point should be capable of facing at an angle to the longitudinal axis of the distal end zone of the catheter, for breaching the wall where required. One convenient way of achieving this angle is to provide the penetrative point on the distal end of a needle of shape memory alloy such as nickel titanium, commonly used for building self-expanding stents. As the needle is advanced from the sheathed to the unsheathed disposition, it can revert to its remembered disposition, somewhat curved, thereby putting the penetrative tip at an angle to the long axis of the catheter.

Conveniently, the catheter will carry an obturator near its distal end, the purpose of the obturator being to expand the hole in the luminal wall made by the broaching means, in preparation for advance through the broached wall of the distal tip of the catheter proper, carrying the shunt. The obturator will likely have an olive shape, more or less atraumatic.

It will be appreciated that the shunt can be deployed analogously to a stent graft, that is to say, by proximal withdrawal of a confining sheath, once the covered stent has been placed by the catheter with its leading end within the venous lumen, its proximal end within the arterial lumen along which the catheter has been advanced, and with its intermediate length portion providing the fistula between the two lumens, and the needle stick zone for subsequently receiving the dialysis needle. After proximal withdrawal of the sheath, the covered stent expands radially and the delivery catheter, including obturator and penetrative point, can be proximally withdrawn through the lumen of the shunt.

The method of creating an arteriovascular fistula, with a shunt, in accordance with the present invention, is characterised by the steps of i) advancing a covered stent along a first lumen of the arteriovenous system ii) tunneling through the wall of said lumen, through intervening tissue, and through the wall of a second lumen of the arteriovenous system to create the fistula, and iii) deploying the stent as a shunt in the fistula.

To enable the advance of the catheter containing the shunt, from the artery across to the vein, it is preferred to use a tunneling device to tunnel between a percutaneous slit adjacent the artery where its wall is to be broached, and a second percutaneous slit adjacent the vein where the lumen wall of the vein is to be broached radially inwardly. With access provided with the two percutaneous slits, a tunneling needle can be advanced form one slit to the other and it may be convenient to use the tunneling needle not only to establish a channel through the tissue to receive the shunt delivery catheter, but also to install temporarily in that channel a sheath through which the delivery catheter can advance, said sheath being withdrawn afterwards, through one of the percutaneous slits.

After installation of the shunt, the various delivery components can all be removed, and the percutaneous slits closed and allowed to heal, so that the individual is left with a subcutaneous prosthesis which is an arteriovenous shunt, and the only percutaneous intervention needed is the temporary passage of the dialysis needle during dialysis procedures as such. After each dialysis treatment, the needle is withdrawn and the puncture site in the skin has the chance to heal.

The puncture site in the shunt does not have the capacity for natural healing but does have a capability for a high degree of sealing, through its natural resilience. The blood tightness of the punctured shunt can be enhanced by the provision of a pillow or pad of gel located between the puncture site of the shunt and the overlying cutaneous layers of natural tissue.

In this way, the present invention offers possibilities to enhance the quality of life of dialysis patients, and reduce the likelihood of percutaneous infection. Furthermore, by sparing arterial and venous wall tissue the trauma of repeated needle sticks, the likelihood of local stenosis of the arterial or venous lumens can be reduced.

For a better understanding of the present invention, and to show more clearly how the same may be carried into effect, reference will now be made, by way of example, to the accompanying drawings.

### BRIEF DESCRIPTION OF THE DRAWINGS

FIG. 1 is a sketch of an arteriovenous shunt,

FIG. 2 is a sketch of an arteriovenous fistula,

FIG. 3 is a sketch of an arteriovenous graft,

FIG. 4 is a schematic diagram of a hemodialysis circuit,

FIG. 5 is a diagram of a first embodiment of shunt according to the present invention,

FIG. 6 is a photograph of the venous end of the shunt of FIG. 5,

FIG. 7 is a photograph of the arterial end of the shunt of FIG. 5,

FIG. 8 shows the distal end of a delivery catheter for the shunt of FIG. 5, within an artificial lumen,

FIG. 9 is a photograph of the FIG. 8 catheter distal end, but with a penetrating point in a penetrating disposition; and

FIG. 10 is a photograph form the side, of the arterial end of the shunt shown diagrammatically in FIG. 5.

### DETAILED DESCRIPTION

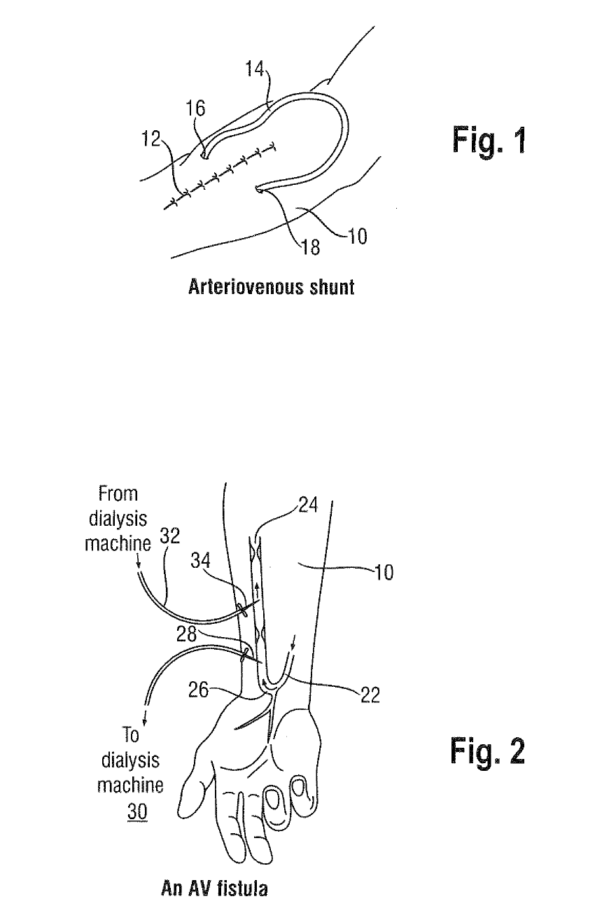

Looking first at FIG. 1, there is shown a human forearm 10 with a stitched together long percutaneous slit 12 and an arteriovenous shunt 14 with one end 16 extending through a percutaneous puncture into an arterial lumen of the forearm, and an opposite end 18 extending through a percutaneous puncture spaced from puncture 16, and into a venous lumen of the forearm. The shunt intermediate length portion is external of the body and therefore available for access by dialysis needles. However, the puncture sites 16 and 18 render the arrangement less than ideal from an infection perspective.

Turning to FIG. 2, there is shown again the human forearm 10 and, diagrammatically, beneath the skin, an arterial lumen 22, a venous lumen 24 and, between them, an arteriovenous fistula 26. A first dialysis needle 28 taps blood for a dialysis machine 30 and a return flow channel 32 from the machine delivers blood back to the patient via a second dialysis needle 34.

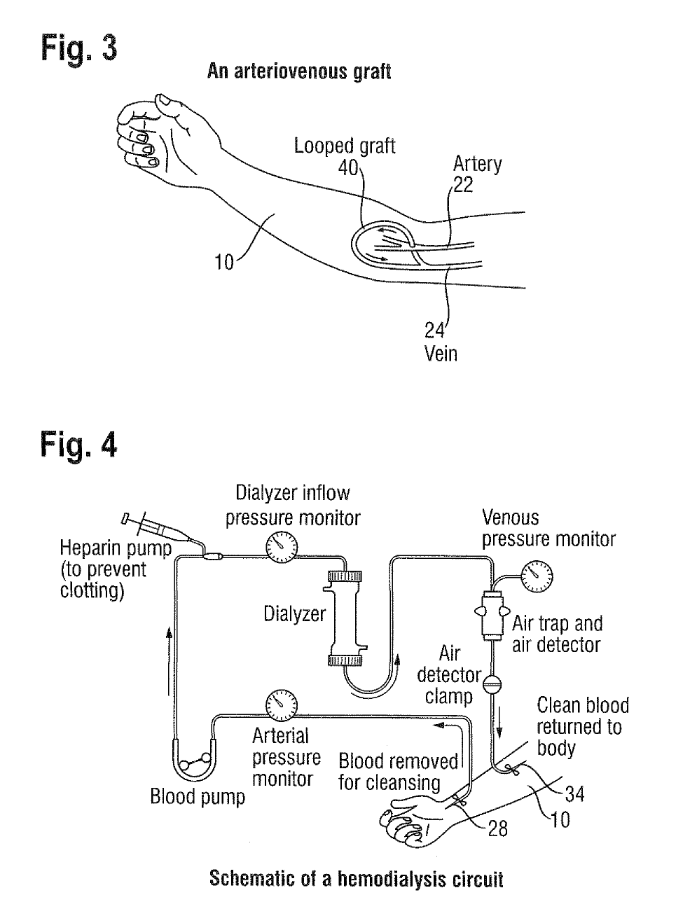

Looking at FIG. 3, we see the same human forearm 10, arterial lumen 22 and venous lumen 24 but, this time, the arteriovenous shunt is provided by a looped graft 40 which is located subcutaneously, and placed by open surgery.

FIG. 4 is included for the sake of completeness. It can be seen as the counterpart to what is shown in FIG. 2, with the same human forearm 10 being represented in the lower right hand corner of the diagram. Needle 28 withdraws blood from the machine, and needle 34 returns it to the body. Various components of the dialysis machine are known to those skilled in the art of access systems for dialysis and, in any case, are labelled in words on the drawing Figure.

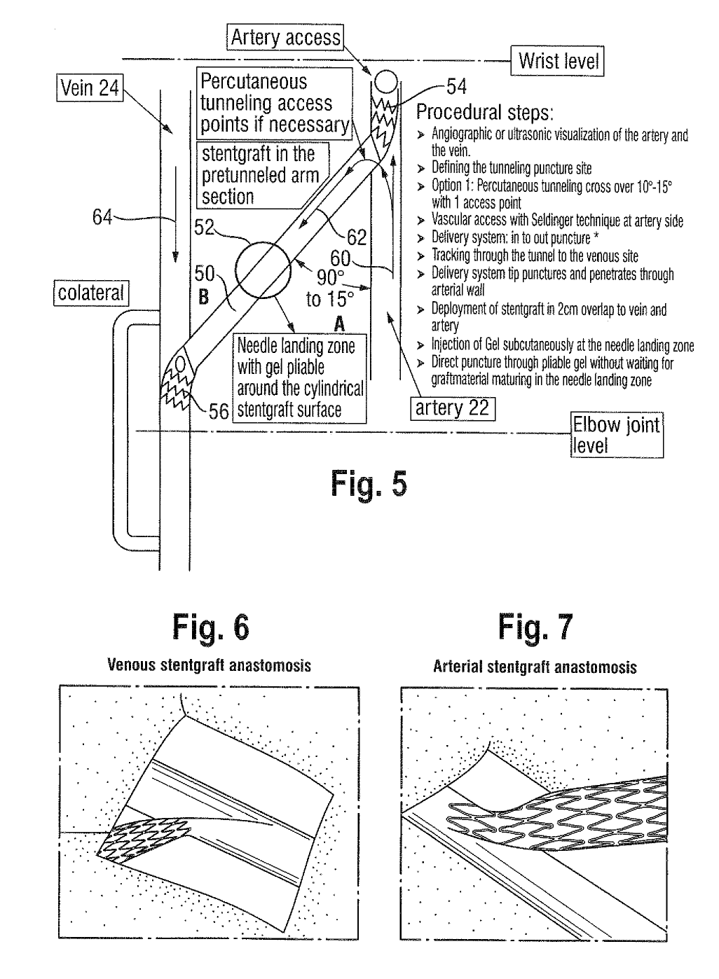

Turning to FIG. 5, this drawing Figure has substantial text content, which is part of the disclosure of the present patent application to enable skilled readers to realise the subject matter of the presently claimed invention. We use again reference 22 to denote the arterial lumen and 24 to indicate the venous lumen which are connected by the shunt. In the present invention, the shunt is in the nature of a stent graft, depicted in FIG. 5 as element 50. It provides a puncture site 52 (otherwise called “needle landing zone”) which can be in one embodiment anywhere along the length of the stent graft between the arterial 22 and venous 24 lumens, or in another embodiment a special zone within that length. In any event, the stent graft exhibits, all the way from one end zone 54 to the other end zone 56, a matrix of stenting struts, as such known to those skilled in the art and to skilled readers. At least in that portion of the length of the stent graft 50 that is not within the arterial lumen 22, nor in the venous lumen 24, the stent matrix is covered, to render it fluid-tight, and the covering in the presently preferred embodiment is of expanded polytetrafluoroethylene.

Important to note in FIG. 5 is the orientation of the shunt with respect to the flow directions of blood in the arterial and venous lumens.

In FIG. 5, arrow 60 shows the flow direction of blood in the arterial lumen 22 and it can be seen that the stent graft shunt has a length direction, arrow 62, that is at an acute angle of between 15° and 90° to the upstream, counter-flow, direction in the artery 22. In consequence, arterial blood flowing along the artery 22 is called upon to turn through an obtuse angle of more than 90° in order to flow along the lumen of the stent graft 50.

By contrast, flow 62 in the stent graft merges with downstream flow 64 in the vein 24 at an acute angle B of less than 90°, more or less complementary to the acute angle A between the stent graft and the upstream direction in the artery. In order to facilitate flow from the artery into the shunt, the covering on the stent graft is cropped back from the arterial end 54 of the stent mesh, along a line that takes a slanting angle with respect to the longitudinal axis 62 of the stent graft, so that the slanting line where the covering ends coincides more or less with the luminal wall of the artery 22. The uncovered stent mesh 54 inside the arterial lumen is hardly any impediment to the flow of blood onward down the arterial lumen, but does serve as a useful anchor for the stent graft shunt within the arterial lumen.

Likewise, at the venous end 56 of the shunt, again, the stent graft covering can be cut back to a line that more or less corresponds to the venous wall, and the uncovered end portion 56 of the stent mesh within the venous lumen again acts as a useful anchor without substantially impeding venous flow onward down the lumen 24.

The photographs of FIGS. 6 and 7 are of artificial lumens made of polymer, but receiving the opposed ends of a stent graft of the general form contemplated for the present invention. It can be seen that the luminal wall of synthetic polymer is resilient enough to squeeze the diameter of the stent graft, so that the diameter of the lumen of the stent graft is locally constricted, where the stent graft passes through the wall of the arterial and venous lumens. However, that restriction is not sufficient to close entirely the lumen of the shunt. Important for the reader to appreciate is that, in this respect, the artificial polymeric lumen is a poor imitation of bodily tissue, which has more capability to accommodate the prosthesis. Over time, the self-expanding stent will expand radially, as the lumen wall tissue (vein and artery) relaxes and undergoes strain, so that there will be little or no local constriction in the shunt flow path through the arterial or venous wall. It is contemplated to use a percutaneous transluminal angioplasty step (familiar as such to skilled readers) to assist in removing the local constriction to the extent judged desirable or necessary.

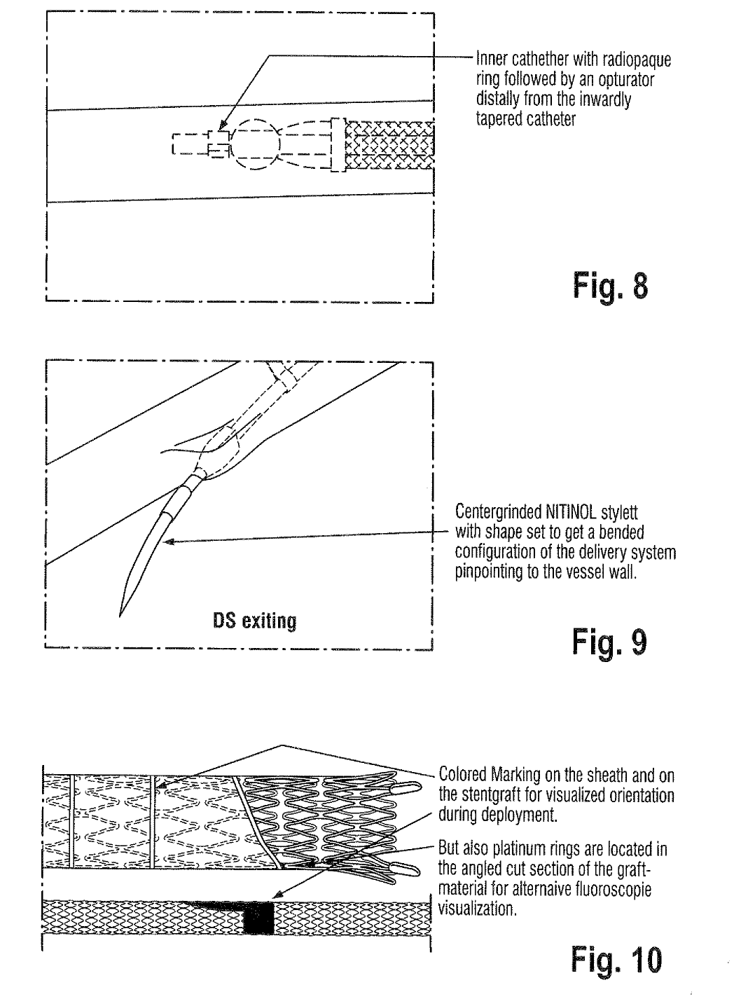

Moving on, to consider FIG. 8, a transparent polymer tube provides a model of an arterial lumen. The photograph in FIG. 8 reveals the details of the distal end of the catheter that is used to deliver the arteriovenous shunt. The open distal end is provided with a radiopaque ring (conveniently of platinum) and immediately proximal of the radiopaque marker ring is an olive-shaped obturator that itself sits immediately distal of a tapered distal end portion of a sheath that confines a radially expansible self-expanding stent graft radially inside the sheath. The sheath is itself provided with a radiopaque marker, again, conveniently, a band of platinum. There is a substantial lumen within the inner tube that carries the obturator. The lumen can accommodate a penetrating means and that, in turn, can define a guidewire lumen.

Moving on to FIG. 9, here with see the same olive-shaped obturator but this has been advanced distally, on the inner tube, so that there is significant axial spacing between it and the radiopaque marker ring on the stent-confining sheath. Furthermore, distal of the obturator, a penetrating point on a nickel titanium shape memory alloy shaft, has been extended distally, from inside the lumen of the inner tube, from a sheathed non-penetrative disposition, into a distally extended, unsheathed, penetrative disposition, in which it has indeed penetrated the wall of the synthetic artery, and has been further distally advanced until the olive-shaped obturator is located within the puncture of the arterial wall made by the penetrating point.

Not shown in FIG. 9 is a pre-prepared tunneled channel from the puncture point of the arterial lumen to an intended puncture point of a venous lumen, along which the NITINOL stylet now advances, until the penetrating point punctures the wall of the venous lumen. It is contemplated that the delivery catheter inner tube that terminates in the penetrating point defines a guidewire lumen and that the guidewire is advanced past the penetrating point into the venous lumen. Then, even after proximal withdrawal of the penetrating point into a sheathed non-penetrating disposition, the delivery catheter can be advanced along the guidewire until the obturator olive has passed through the puncture in the wall of the venous lumen and enters the lumen.

At this point, the way is clear for the delivery catheter to be advanced, far enough to advance the leading end of the stent graft, still within the confining sheath of the catheter, into the venous lumen. With percutaneous slits, previously used to create the tunneled channel for the shunt catheter, progress of the stent graft within its confining sheath can be observed directly visually, and coloured or other visually recognisable markers on the stent graft can be viewed through the slits to ensure that the desired length of the respective end portions of the stent graft are safely located inside the lumen of the artery and vein respectively. When the stent graft is exactly in position, the sheath radially confining the stent graft can be progressively withdrawn proximally analogously to the well-known way of deploying a stent graft or bare stent, until the sheath is fully proximal of the proximal end of the stent in the arterial lumen. After that, the inner shaft of the delivery catheter, complete with obturator olive and marker band and penetrating point, can all be withdrawn through the lumen of the expanded stent graft.

Readers will grasp that the proximal end of the catheter will have a hand-held actuator (as is conventional for delivery of stent grafts trans-luminally). However, the hand unit for the present invention must be capable of rotating the catheter about its long axis to an extent such that the penetrating point can point in the right direction, and the shunt can be given the rotational orientation that will line up the slanting line at the end of the stent covering with the wall of the arterial and, respectively, venous lumens at each end of the shunt.

Once the stent graft is in position, a special gel can be injected through the skin, or introduced through one of the percutaneous slits, to sit as a pad or cushion between the stent graft needle landing zone and the skin layers of the patient. The gel cushions the stent graft and helps to seal it after successive penetrations by the needles of a dialysis machine.

As noted in the text on FIG. 5, the system described above has the potential to permit dialysis with a dialysis machine, more or less immediately after installation of the shunt, without having to wait, as conventionally, for the graft material to mature.

Bio-active agents can be added to the prosthesis (e.g., either by a coating or via a carrier medium such as resorbable polymers) for delivery to the host's vessel or duct. The bio-active agents may also be used to coat the entire stent. A material forming the stent or coupled to the stent may include one or more (a) non-genetic therapeutic agents, (b) genetic materials, (c) cells and combinations thereof with (d) other polymeric materials.

(a) Non-genetic therapeutic agents include anti-thrombogenic agents such as heparin, heparin derivatives, urokinase, and PPack (dextrophenylalanine proline arginine chloromethylketone); anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; anti-coagulants, an RGD peptide-containing compound, heparin, antithrombin compounds, platelet receptor antagonists, anti-thrombin antibodies, anti-platelet receptor antibodies, aspirin, prostaglandin inhibitors, platelet inhibitors and tick antiplatelet peptides; vascular cell growth promoters such as growth factor inhibitors, growth factor receptor antagonists, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; and agents which interfere with endogenous vascoactive mechanisms.

(b) Genetic materials include anti-sense DNA and RNA, DNA coding for, anti-sense RNA, tRNA or rRNA to replace defective or deficient endogenous molecules, angiogenic factors including growth factors such as acidic and basic fibroblast growth factors, vascular endothelial growth factor epidermal growth factor, transforming growth factor alpha and beta, platelet-derived endothelial growth factor, platelet-derived growth factor, tumor necrosis factor alpha, hepatocyte growth factor and insulin like growth factor, cell cycle inhibitors including CD inhibitors, thymidine kinase (“TK”) and other agents useful for interfering with cell proliferation the family of bone morphogenic proteins (“BMP's”), BlVfiP-2, BMP-3, BMP-4, BNP-5, BMP-6 (Vgr-1), BMP-7 (OP-1), BMP-8, BMP-9, BMP10, BMP-1, BMP-12, BMP-13, BMP-14, BMP-15, and BMP-16. Desirable BNP's are any of BMP-2, BMP-3, BMP-4, BMP-5, BMP-6 and BNP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

(c) Cells can be of human origin (autologous or allogeneic) or from an animal source (xenogeneic), genetically engineered if desired to deliver proteins of interest at the deployment site. The cells may be provided in a delivery media. The delivery media may be formulated as needed to maintain cell function and viability.

(d) Suitable polymer materials as a coating or the base material may include polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof, coatings from polymer dispersions such as polyurethane dispersions (for example, BAYHDROL® fibrin, collagen and derivatives thereof, polysaccharides such as celluloses, starches, dextrans, alginates and derivatives, hyaluronic acid, squalene emulsions. Polyacrylic acid, available as HYDROPLUS® (Boston Scientific Corporation, Natick, Mass.), and described in U.S. Pat. No. 5,091,205, the disclosure of which is hereby incorporated herein by reference, is particularly desirable. Even more desirable is a copolymer of polylactic acid and polycaprolactone.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

1. A covered radially-expansible stent, capable of being advanced along a bodily lumen, and capable of performing as an arteriovenous shunt, and with a covering that stops short of one end of the stent at a line that slants to the longitudinal axis of the stent at an angle intermediate between 0° and 90°.

2. The stent according to claim 1, wherein the covering of the stent comprises ePTFE.

3. The stent according to claim 1, further comprising at least one radiopaque marker.

4. The stent according to claim 3, comprising at least one visual marker adapted to be viewed directly by the unaided human eye.

5. The stent according to claim 4, wherein the visual marker has a colour different from adjacent areas of the stent.

6. A system for creating an arteriovenous fistula with a shunt, comprising:
a catheter for advancing a covered stent carried near the distal end of the catheter along a bodily lumen into a position in which the stent can function as an arteriovenous shunt between a first and a second bodily lumen, said catheter including means to broach radially outwardly a wall of said first lumen and radially inwardly a wall of said second lumen, and then deploy the stent.

7. The system according to claim 6, wherein the broaching means is a penetrating point at the distal end of the catheter.

8. The system according to claim 7, including means to move the point between a distally advanced unsheathed penetrative disposition and a proximally retracted sheathed non-penetrative disposition.

9. The system according to claim 7, wherein the point is capable of facing at an angle to the longitudinal axis of the distal end zone of the catheter, for broaching said wall of said first lumen.

10. The system according to claim 6, including an obturator at the distal end of the catheter, distal of the location near the distal end where the stent is carried.

11. The system according to claim 10, wherein the obturator has an olive shape.

12. A method of creating an arteriovascular fistula with a shunt, comprising:
i) advancing a covered stent along a first lumen of the arteriovenous system;
ii) tunneling through the wall of said first lumen, through intervening tissue, and through the wall of a second lumen of the arteriovenous system to create the fistula; and
iii) deploying the stent as a shunt in the fistula.

13. The method according to claim 12, wherein the advancing step includes providing a delivery catheter carrying a self-expanding stent having a covering that stops short of both a proximal and a distal end thereof, the terminal ends of the covering angled obliquely with respect to the longitudinal axis of the stent, further comprising the step of rotating the stent to align the terminal ends with the wall of the first lumen and second lumen of the arteriovenous system.

14. The method according to claim 13, wherein the delivery catheter includes an inner tube having a lumen in which is slidably disposed a tunneling apparatus formed from a nickel titanium shape memory alloy and including an elongate shaft and a penetrating distal tip, the tunneling step comprising sliding the penetrating tip out of the inner tube lumen and into contact with the wall of the first lumen, the penetrating tip transitioning from a first configuration substantially parallel to a longitudinal axis of the inner tube lumen to a second configuration at an angle to the longitudinal axis of the inner tube lumen.

15. The system according to claim 6, further comprising a covered stent carried near the distal end of the catheter, the stent having a covering that stops short of both a proximal and a distal end thereof, the terminal ends of the covering angled obliquely with respect to the longitudinal axis of the stent.

16. The system according to claim 15, wherein the means to broach comprises a penetrating point at the end of an elongate shaft, the point and shaft formed from a nickel titanium shape memory alloy.

17. The system according to claim 16, wherein the penetrating point has a first configuration substantially parallel to the longitudinal axis of the catheter when the point is within the catheter, and a second configuration at an angle to the longitudinal axis of the catheter when the point is outside of the catheter, the transition from the first configuration to the second configuration resulting from a change in temperature.

18. The system according to claim 17, wherein the catheter comprises an inner tube and an outer sheath, the stent held between the inner tube and outer sheath, the penetrating point and shaft slidably disposed in a lumen of the inner tube, a distal end of the inner tube extending distally of a distal end of the outer sheath.

19. The system according to claim 18, wherein the inner tube includes an obturator positioned distally of the distal end of the outer sheath, a radiopaque ring positioned on the inner tube distally of the obturator.

20. The system according to claim 15, wherein the penetrating point and elongate shaft include a guidewire lumen.
